(19) United States
(12) Patent Application Publication
(10) Pub. No.: US 2011/0137169 A1
(43) Pub. Date: Jun. 9, 2011

(54) MEDICAL IMAGE PROCESSING APPARATUS, A MEDICAL IMAGE PROCESSING METHOD, AND ULTRASONIC DIAGNOSIS APPARATUS

(75) Inventors: Kazuya AKAKI, Tochigi-ken (JP); Masaru Ogasawara, Tochigi-ken (JP); Takayuki Gunji, Tochigi-ken (JP); Satoshi Matsunaga, Tochigi-ken (JP); Yutaka Kobayashi, Tochigi-ken (JP)

(73) Assignees: KABUSHIKI KAISHA TOSHIBA, Tokyo (JP); TOSHIBA MEDICAL SYSTEMS CORPORATION, Otawara-shi (JP)

(21) Appl. No.: 12/959,847

(22) Filed: Dec. 3, 2010

(30) Foreign Application Priority Data
Dec. 9, 2009 (JP) P2009-279732
Dec. 1, 2010 (JP) P2010-268483

(51) Int. Cl. A61B 8/14; G09G 5/00

(52) U.S. Cl. 600/443; 345/629

(57) ABSTRACT

A medical image processing apparatus that acquires a medical image and additional information associated with the medical image. The medical image processing apparatus reduces a display size of the medical image using a first size-reduction rate, and reduces a display size of the additional information using a second size-reduction rate. The medical image processes apparatus then combines the size-reduced medical image and the size-reduced additional information into a thumbnail composite image, and displays the thumbnail composite image.

### CROSS-REFERENCE TO RELATED APPLICATIONS

This application is based upon and claims the benefit of priority from prior Japanese Patent Application No. 2009-279732, filed Dec. 9, 2009, the entire contents of which are incorporated herein by reference.

### FIELD

Embodiments described herein relate generally to a medical image processing apparatus, a medical image processing method, and an ultrasonic diagnosis apparatus that shows thumbnail images with clinical information.

### BACKGROUND

A medical image processing apparatus treats images that are used for medical diagnosis. The term “medical image processing apparatus” includes various apparatuses that treat medical images, such as X-ray, CT (Computed Tomography), MRI (Magnetic Resonance Imaging), PET (Positron Emission Tomography), endscopic instruments, and workstations for processing medical images using PACS (Picture Archiving and Communication System).

An ultrasonic diagnosis apparatus is an example of a medical image processing apparatus that allows one to display, in real time, how the heart beats or the fetus moves. In addition, an ultrasonic diagnosis apparatus is free from the influences of exposure using X-rays, and thus is high in safety and can be used in an obstetrical setting, for medical examination of breast cancer, for home medical care, etc.

When an ultrasonic diagnosis apparatus generates medical images, at first, the ultrasonic diagnosis apparatus gets a medical image by ultrasonic scanning and stores that medical image in a storage unit installed on the ultrasonic diagnosis apparatus. Next, the ultrasonic diagnosis apparatus obtains clinical information about the medical image. For example, clinical information can show a time sequence of heartbeats. Next, the ultrasonic diagnosis apparatus generates a full-sized composite image by combining the clinical information and the medical image as a full-sized medical image.

After generating the full-sized composite image, the ultrasonic diagnosis apparatus displays the full-sized composite image on a monitor. In addition, the ultrasonic diagnosis apparatus displays a thumbnail image, which is reduced in size compared to the full-sized composite image. An operator of the ultrasonic diagnosis apparatus uses thumbnail images to find a full-sized composite image. FIG. 14 shows an example of thumbnail images on a display.

When an operator tries to get medical images of the heart, the ultrasonic diagnosis apparatus scans the heart during some cardiac beats, and then generates a series of medical images. Next, the ultrasonic diagnosis apparatus gets additional information about the series of medical images. Next, the ultrasonic diagnosis apparatus generates a series of full-sized composite images by combining the medical images and the additional information. Next, the ultrasonic diagnosis apparatus reduces a size of each of the full-sized composite images, and generates a series of thumbnail images. The ultrasonic apparatus displays the series of thumbnail images. When the operator selects one image from the series of thumbnail images, the ultrasonic apparatus displays the full-sized composite image corresponding to the selected image.

The ultrasonic diagnosis apparatus described above has several problems. In particular, the ultrasonic diagnosis apparatus displays the thumbnail images with additional information of reduced size. The size-reduction rate of the additional information is the same as that of the thumbnail image. FIG. 14 shows the additional information having reduced size in the thumbnail image. Clearly, the operator cannot read the additional information in the thumbnail images.

When the operator tries to read the additional information, the operator has to select each thumbnail image in order to display the full-sized composite image with the full-sized additional information. Such operations take time and require additional labor for the operator.

### BRIEF DESCRIPTION OF THE VIEWS OF THE DRAWINGS

FIG. 1 is a block diagram of a medical image processing apparatus as an ultrasonic diagnosis apparatus according to an embodiment;

FIG. 2 is a flowchart showing a processing procedure generating thumbnail composite images;

FIG. 3 shows a full-sized composite image including an ultrasonic image as a full-sized image and an ECG waveform over one cardiac beat;

FIG. 4 shows a thumbnail composite image including an ultrasonic image as a thumbnail image and an ECG waveform over one cardiac beat;

FIG. 5 shows a full-sized composite image including of an ultrasonic image as a full-sized image and an ECG waveform over several cardiac beats;

FIG. 6 shows a thumbnail composite image including an ultrasonic image as thumbnail an image and text representing a time phase over cardiac beats;

FIG. 7 shows a full-sized composite image including an ultrasonic image as a full-sized image and an ECG waveform over several cardiac beats and associated findings;

FIG. 8 shows a thumbnail composite image including an ultrasonic image as a thumbnail image and associated findings;

FIG. 9 shows a full-sized composite image including an ultrasonic image as a full-sized image and an ECG waveform and measured values of a Doppler waveform;

FIG. 10 shows a thumbnail composite image including an ultrasonic image as a thumbnail image and measured values of a Doppler waveform;

FIG. 11 shows a full-sized composite image including an ultrasonic image as a full-sized image and a pictogram;

FIG. 12 shows a thumbnail composite image including an ultrasonic image as a thumbnail image and a pictogram;

FIG. 13 is a flowchart showing a processing procedure generating thumbnail composite images in a fifth modified example;

FIG. 14 shows a display example of displaying thumbnail composite images;

FIG. 15 shows a display example of displaying thumbnail images; and

FIG. 16 is a block diagram of a medical image processing apparatus as a workstation according to an embodiment.

### DETAILED DESCRIPTION

A medical image processing apparatus according to one embodiment comprises a medical image acquisition unit configured to acquire a medical image; an additional information acquisition unit configured to acquire additional information associated with the medical image; a thumbnail image composite unit configured to reduce a display size of the medical image using a first size-reduction rate, to reduce a display size of the additional information using a second size-reduction rate, and to combine the size-reduced medical image and the size-reduced additional information into a thumbnail composite image; and a display unit configured to display the thumbnail composite image.

An embodiment will be described below with reference to the views of the accompanying drawings. Note that the same reference numerals denote constituent elements having almost the same functions and arrangements, and a repetitive description will be made only when required.

Certain embodiments described herein will describe a medical image processing apparatus as an ultrasonic diagnosis apparatus. But an ultrasonic diagnosis apparatus is only an example of a medical image processing apparatus, and the described embodiments are not limited to an ultrasonic diagnosis apparatus. Other examples of a medical image processing apparatus include, for example, an X-ray, a CT (Computed Tomography), an MRI (Magnetic Resonance Imaging), PET (Positron Emission Tomography), endscopic instruments, and workstations for treating medical images and processing images using PACS (Picture Archiving and Communication System).

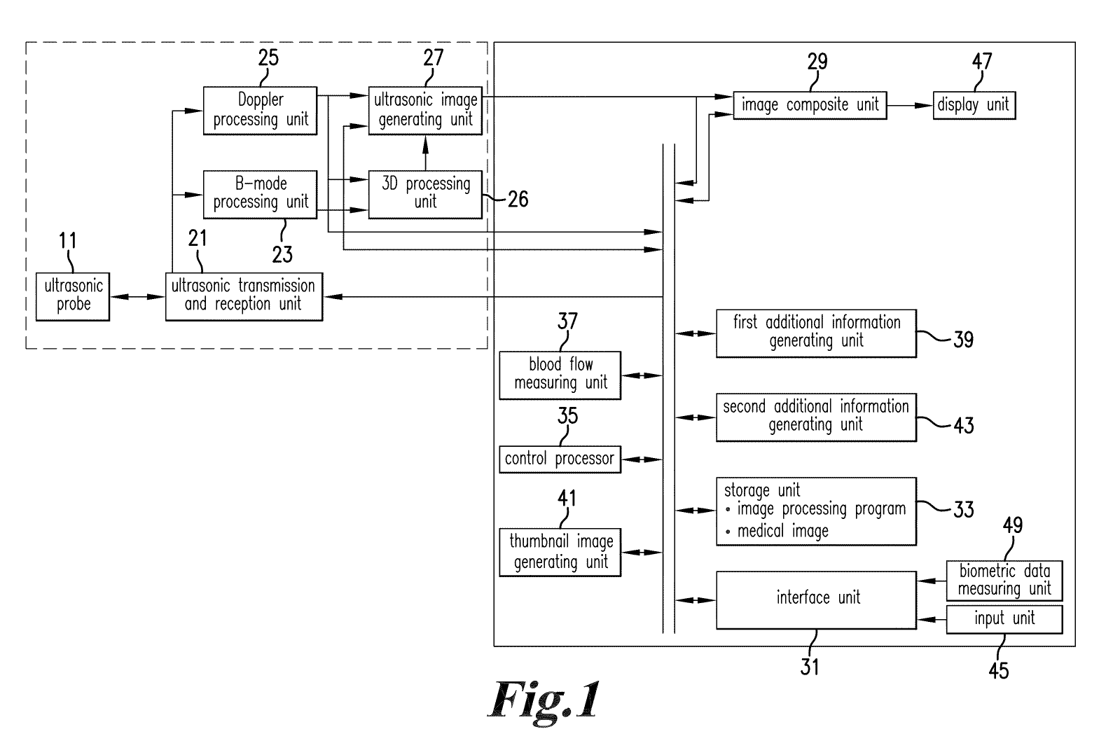

FIG. 1 is a block diagram showing the arrangement of an ultrasonic diagnosis apparatus according to this embodiment. As shown in FIG. 1, the ultrasonic diagnosis apparatus includes an ultrasonic probe 11, an ultrasonic transmission and reception unit 21, a B-mode processing unit 23, a Doppler processing unit 25, a 3D processing unit 26, an ultrasonic image generating unit 27, an image composite unit 29, an interface unit 31, a storage unit 33, a control processor 35, a blood flow measuring unit 37, a first additional information generating unit 39, a thumbnail image generating unit 41, a second additional information generating unit 43, an input unit 45, a display unit 47, and a biometric data measuring unit 49. The function of each constituent element will be described below.

The ultrasonic probe 11 includes (1) a plurality of piezoelectric transducers that generate ultrasonic waves based on driving signals from the ultrasonic transmission and reception unit 21, and convert reflected waves from an object into electrical signals, (2) a matching layer provided for the piezoelectric transducers, and (3) a backing member that prevents ultrasonic waves from propagating backward from the piezoelectric transducers. The transducers are arranged in rows at one side of the ultrasonic probe 11, and a row of transducers forms one channel. Note that the ultrasonic probe 11 may have one channel of transducers and scan two-dimensional areas, or may have multiple channels of transducers and scan three-dimensional areas, or may have one channel of transducers that can move using motors and scans three-dimensional areas.

The ultrasonic transmission and reception unit 21 includes a trigger generating circuit, a delay circuit, and a pulsar circuit (none of which are shown). The pulsar circuit repetitively generates rate pulses for the formation of transmission ultrasonic waves at a predetermined rate frequency. The delay circuit gives each rate pulse a delay time necessary to focus an ultrasonic wave into a beam and to determine transmission directivity for each channel. The trigger generating circuit applies a driving pulse to the ultrasonic probe 11 at a timing based on this rate pulse. The ultrasonic transmission and reception unit 21 also includes an amplifier circuit, an A/D converter, and an adder (none of which are shown). The amplifier circuit amplifies an echo signal received via the ultrasonic probe 11 for each channel. The A/D converter gives the amplified echo signals delay times necessary to determine reception directivities. The adder then performs addition processing for the signals.

The B-mode processing unit 23 receives an echo signal from the ultrasonic transmission and reception unit 22, and performs logarithmic amplification, envelope detection processing, etc. for the signal to generate B-mode data whose signal intensity is expressed by a luminance level. The B-mode processing unit 23 outputs the B-mode data to the ultrasonic image generating unit 27.

The Doppler processing unit 24 frequency-analyzes velocity information from the echo signal received from the ultrasonic transmission and reception unit 22 to extract a blood flow, tissue, and contrast medium echo component using the Doppler effect. The Doppler processing unit 24 obtains Doppler-data that represents blood flow information such as an average velocity, variance, and power at multiple points. The Doppler processing unit 24 outputs the Doppler-data to the ultrasonic image generating unit 27.

The 3D processing unit 26 generates volume data by mapping the B-mode data or the Doppler data to three-dimensional coordinates. The 3D processing unit 26 generates volume data when the ultrasonic probe 21 scans three-dimensional areas. The 3D processing unit 26 outputs the volume data to the ultrasonic image generating unit 27.

The ultrasonic image generating unit 27 generates medical images as a full-sized image by using the B-mode data or the Doppler-data or the volume data. For example, the ultrasonic image generating unit 27 generates a two-dimensional B-mode image by mapping the B-mode data to two-dimensional coordinates. As another example, the ultrasonic image generating unit 27 generates a two-dimensional Doppler image by mapping the Doppler data to two-dimensional coordinates. As another example, the ultrasonic image generating unit 27 generates a two-dimensional projection image by projecting the volume data from an appropriate projection point. As another example, the ultrasonic image generating unit 27 generates a two-dimensional planner image by extracting data that exists in a same plane in the three-dimensional area of the volume data, and mapping the extracted data to two-dimensional coordinates. As another example, the ultrasonic image generating unit 27 generating a two-dimensional rendered image by processing the volume rendering to the volume data. Note that this embodiment describes “raw data” as the generic term of the B-mode data, the Doppler data, and the volume data. Further, note that the ultrasonic image generating unit 27 may generate a moving image of the B-mode image, the Doppler image, and the 3D image by using the raw data obtained by a sequential scan.

Note that the medical images generated by the ultrasonic image generating unit 27 are not limited to the above description. For example, the ultrasonic image generating unit 27 may generate a TH (Third Harmonic) image by extracting harmonic data from echo data. As another example, the ultrasonic image generating unit may generate an M-mode image by mapping the temporal variation of echo data in a certain area.

The interface unit 31 outputs or inputs signals with external storage units that are connected to the ultrasonic diagnosis apparatus, and the biometric data measuring unit 49. The interface unit 31 can transfer data such as the medical image as a full-sized image and the first additional information to the external apparatuses and storage units connected to the ultrasonic diagnosis apparatus through the network.

The blood flow measuring unit 37 generates trace waveforms by tracing the maximum velocity using Doppler data. The blood flow measuring unit 37 estimates a time phase of the early diastolic flow (E wave) and the arterial contraction flow (A wave) using the trace waveform and the electrocardiographic (ECG) waveform that are outputted by the biometric data measuring unit 49. The blood flow measuring unit 37 measures the DcT (Deceleration Time) and E/A (amplitude ratio of E wave to A wave).

The first additional information generating unit 39 generates the first additional information that represents clinical information about the medical image. For example, clinical information is an ECG waveform, a phonocardiographic (PCG) waveform, a sphygmographic waveform, or an pneumogram. As another example, clinical information is time information that indicates when the medical image was obtained. As another example, clinical information is measured data that is obtained from the blood flow measuring unit 37. As another example, clinical information is a measuring parameter used by the blood flow measuring unit 37. As another example, clinical information is a pictogram (body mark) that represents a patient's body simplistically. As another example, clinical information is text that represent the clinical findings from the medical image.

The thumbnail image generating unit 41 generates thumbnail images by reducing the size of the medical image that is obtained from the ultrasonic image generating unit 27. The thumbnail image generating unit 41 reduces a display size of the medical image using a first size-reduction rate. Note that the thumbnail image generating unit 41 may generate the thumbnail image by using a medical image that is obtained from the external apparatus or by using a medical image that is stored by the storage unit 33. Note that if the medical image includes a moving image, the thumbnail image generating unit 41 generates the thumbnail image as the moving image.

The second additional information generating unit 43 generates second additional information by using the first additional information obtained from the first additional information generating unit 39 or the storage unit 33. The second additional information generating unit 43 extracts a section of the first additional information and reduces a display size of the extracted section of the first additional information using a second size-reduction rate. Note that the second size-reduction rate has a different value than the first size-reduction rate. Thus, the display magnification of the thumbnail image reduced in size using the first size-reduction rate is relatively low compared to the display magnification of the second additional information reduced in size using the second size-reduction rate. Note that the second size-reduction rate can be 100%. That is, the second additional information generating unit 43 may generate the second additional information at a same display magnification of the first additional information.

The second additional information simplistically represents clinical information included in the first additional information. The second additional information generating unit 43 generates the second additional information when the storage unit 33 stores the medical image. Note that the second information generating unit 43 may generate the second additional information when the storage unit 33 outputs the medical image. The timing of generating the second additional information is configured by an input operation from the input unit 45.

The control processor 35 functions as an information processing apparatus and controls the operation of the main body of the ultrasonic diagnosis apparatus. In particular, the control processor 35 outputs a dedicated program for generating the medical image and generating the additional information from the storage unit 33, expands the program in the memory of the processor, and executes computation/control, etc. associated with various kinds of processing.

The image composite unit 29 generates a full-sized composite image by combining the medical image as a full-sized image obtained from the ultrasonic image generating unit 27 and the first additional information obtained from the first additional information generating unit 39. The image composite unit 29 displays the full-sized composite image on the display unit 47. The image composite unit 29 also generates a thumbnail composite image by combining the thumbnail image obtained from the thumbnail image generating unit 41 and the second additional information obtained from the second additional information generating unit 43. The image composite unit 29 displays the thumbnail composite image on the display unit 47.

The storage unit 33 stores a control program for executing image generation and display processing, the medical image as a full-sized image, the medical image as a thumbnail image, the raw data, the first additional information, the second additional information, the full-sized composite image, and the thumbnail composite image. The storage unit 33 stores the dedicated program for generating the second additional information by extracting information from the first additional information, and the dedicated program for generating the thumbnail composite image by combining the thumbnail image and the second additional information. Data in the storage unit 33 can be transferred to the external apparatus or the external storage devices via the interface unit 31.

The biometric data measuring unit 49 comprises an electrocardiographic monitor, a phonocardiographic monitor, sphygmographic monitor, a respiration sensor, and an apparatus measuring biometric data. The electrocardiographic monitor generates an ECG waveform by measuring temporal variability of the electrical phenomenon of the heart. The electrocardiographic monitor outputs the ECG waveform as the first additional information to the first additional information generating unit 39 or to the storage unit 33. The phonocardiographic monitor generates the PCG waveform by measuring temporal variability of the vibrating sound of the heart. The phonocardiographic monitor outputs the PCG waveform as the first additional information to the first additional information generating unit 39 or to the storage unit 33. The sphygmographic monitor generates the sphygmographic waveform by measuring temporal variability of the blood flow pressure in a blood vessel. The sphygmographic monitor outputs the sphygmographic waveform as the first additional information to the first additional information generating unit 39 or the storage unit 33. The respiration sensor generates a respiration waveform by measuring the temporal variability of the stomach movement. The respiration sensor outputs the respiration waveforms as the first additional information to the first additional information generating unit 39 or to the storage unit 33.

The input unit 45 connected to the interface unit 31 includes various types of switches and buttons, a trackball, a mouse, and a keyboard that are used to input, to the control processor 35, various types of instructions, conditions, various types of image quality condition setting instructions, etc. from the operator. When, for example, the operator operates the FREEZE button of the input unit 45, the transmission and reception of ultrasonic waves are terminated, and the ultrasonic diagnosis apparatus is set in a temporary stop state.

The display unit 47 displays the full-sized composite image and the thumbnail composite image based on signals outputted by the ultrasonic image generating unit 27.

The Additional Information Generating Function

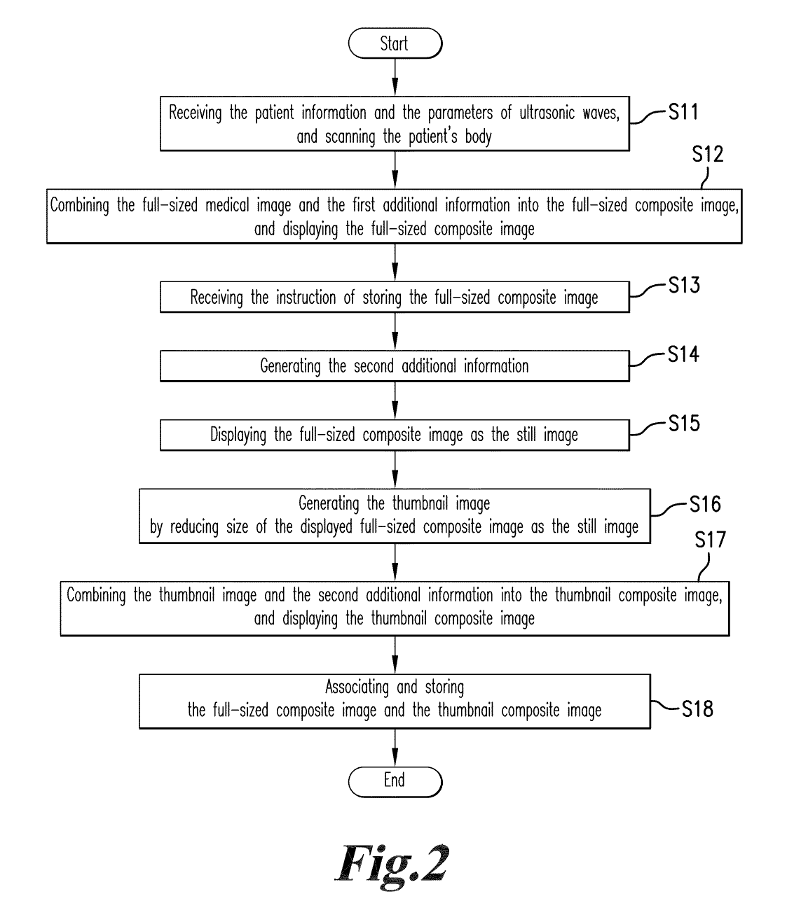

The additional information generating function of the ultrasonic diagnosis apparatus will be described next. FIG. 2 is a flowchart showing a processing procedure of generating the first and the second additional information. This embodiment describes an example in which the ultrasonic image generating unit 27 generates the B-mode image synchronized with an ECG waveform, and the image composite unit 29 generates the full-sized composite image and the thumbnail composite image of the B-mode image. Note that this embodiment shows only an example, so the ultrasonic image generating unit 27 may generate the Doppler image, M-mode image, the TH image, the 3D image, and the moving image as the medical image. Further, the ultrasonic image generating unit 27 may generate the medical image synchronized with the PCG waveform, the sphygmographic waveform, or the respiration waveform. Further, the ultrasonic image generating unit 27 may generate the medical image without any synchronization.

At first, the operator of the ultrasonic diagnosis apparatus operates the input unit 45 for setting the patient information, the transmission and reception parameters, the generating timing of the thumbnail composite image, the first size-reduction rate of the thumbnail image, and the second size-reduction rate of the second additional information. The storage unit 33 stores the patient information, the transmission and reception parameters, the generating timing, the first size-reduction rate, and the second size-reduction rate.

After setting the parameters, the operator moves the ultrasonic probe 11 to the patient's body. The control processor 35 starts transmission and reception of the ultrasonic waves during a time in which the heart makes more than one beat (Step 11). The ultrasonic transmission and reception unit 21 outputs the echo signal to the B-mode processing unit 23. The B-mode processing unit 23 generates the B-mode data by using the echo signals, and outputs the B-mode data to the ultrasonic image generating unit 27. The ultrasonic image generating unit 27 generates the medical image as a full-sized image. Further, the biometric data measuring unit 49 outputs the ECG waveform as the first additional information to the first additional information generating unit 39.

After outputting the medical image as a full-sized image and outputting the first additional information, the image composite unit 29 generates the full-sized composite image by combining the medical image as a full-sized image with the first additional information. The image composite unit 29 displays the full-sized composite image on the display unit 47 in real-time (Step 12).

When the control processor 35 receives an order for storing the full-sized composite image to the storage unit 33 from the input unit 45 (Step 13), the second additional information generating unit 43 generates the second additional information by reducing a size of the first additional information using the second size-reduction rate (Step 14).

Note that the second additional information generating unit 43 may generate the second additional information when the control processor 35 receives the order for outputting the full-sized composite image from the storage unit 33. Further, the second additional information generating unit 43 may generate the second additional information at a given timing given by the input unit 45.

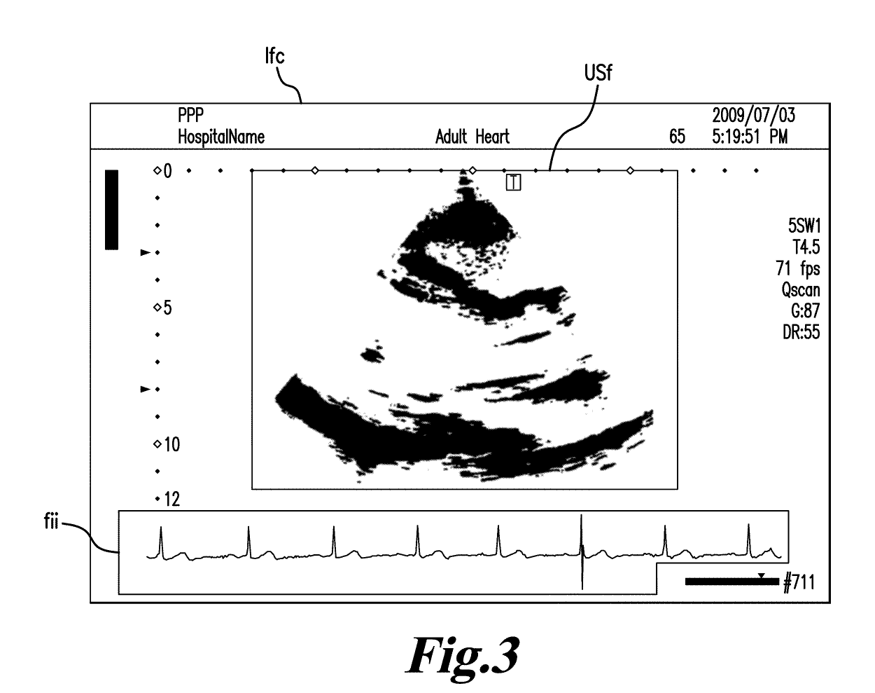

When the storage unit stores the full-sized composite image, the display unit 47 displays the full-sized composite image as a still image (Step 15). FIG. 3 shows the example of the displayed full-sized composite image on the display unit 47. In FIG. 3, Ifc represents the full-sized composite image, USf indicates the medical image as the full-sized image, and flu indicates the first additional information.

When the storage unit stores the full-sized composite image, the thumbnail image generating unit 41 generates the thumbnail image by reducing a size of the medical image as a full-sized image using the first size-reduction rate (Step 16). After generating the thumbnail image, the image composite unit 29 generates the thumbnail composite image by combining the thumbnail image and the second additional information. The image composite unit 29 displays the thumbnail composite image on the display unit 47 (Step 17).

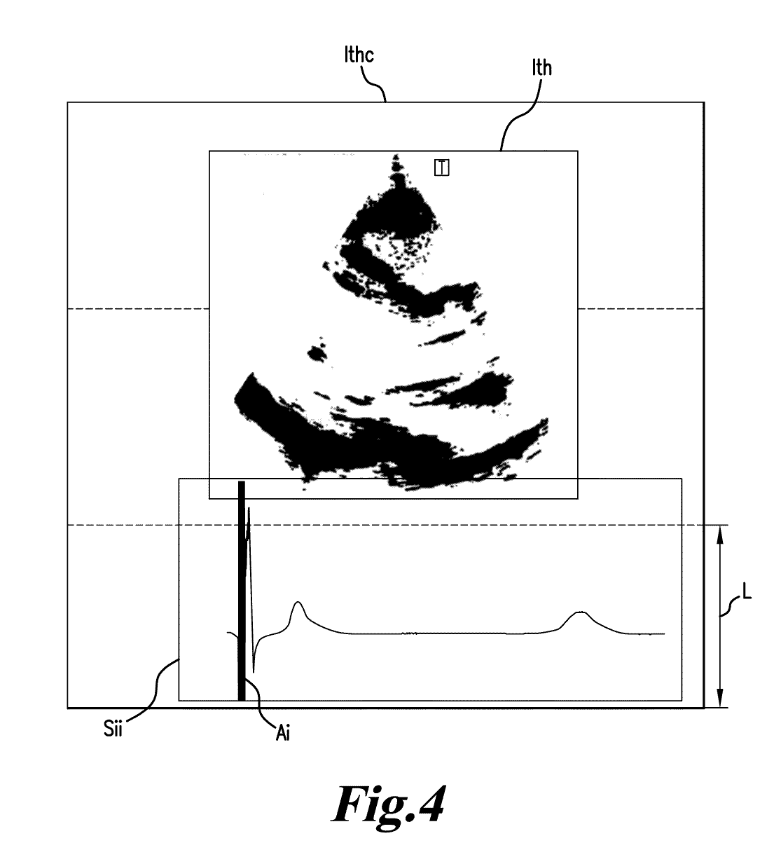

FIG. 4 shows the example of the displayed thumbnail composite image on the display unit 47. In FIG. 4, Ithc indicates the thumbnail composite image, Sii indicates the second additional information, L indicates one-third point to the lateral direction of the thumbnail composite image, and Ai indicates the first additional information. In FIG. 4, the second additional information occupies the lower one-third area of the thumbnail composite image. Note that the display size of the second additional image can be changed by the input operation of the input unit 45.

After displaying the thumbnail composite image, the control processor 35 stores the full-sized composite image and the thumbnail composite image to the storage unit 33 (Step 18).

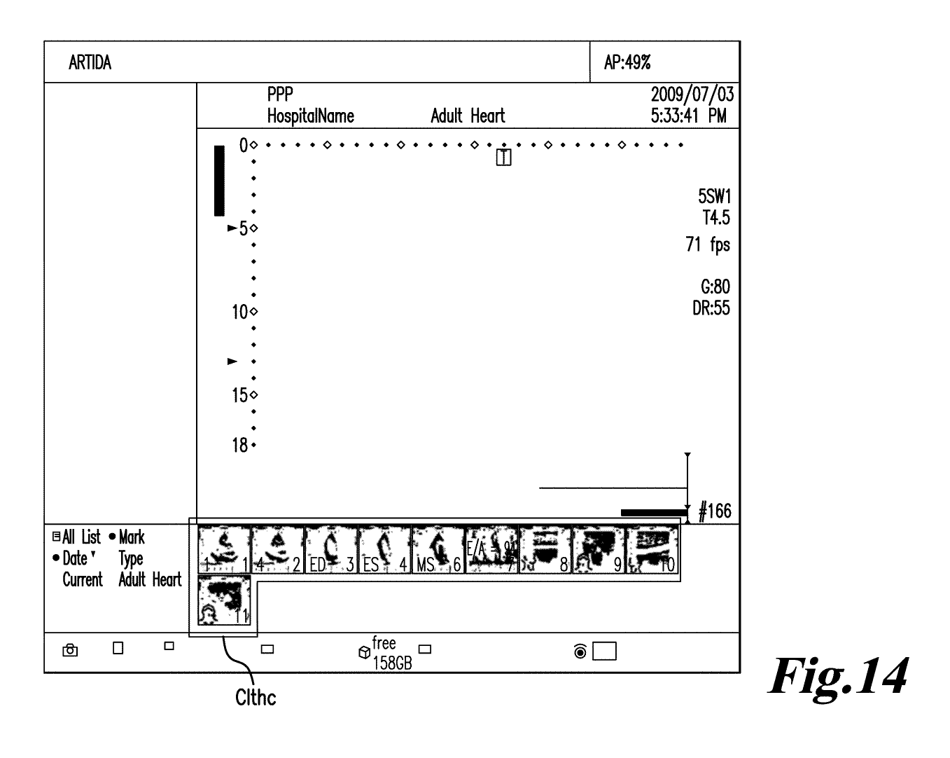

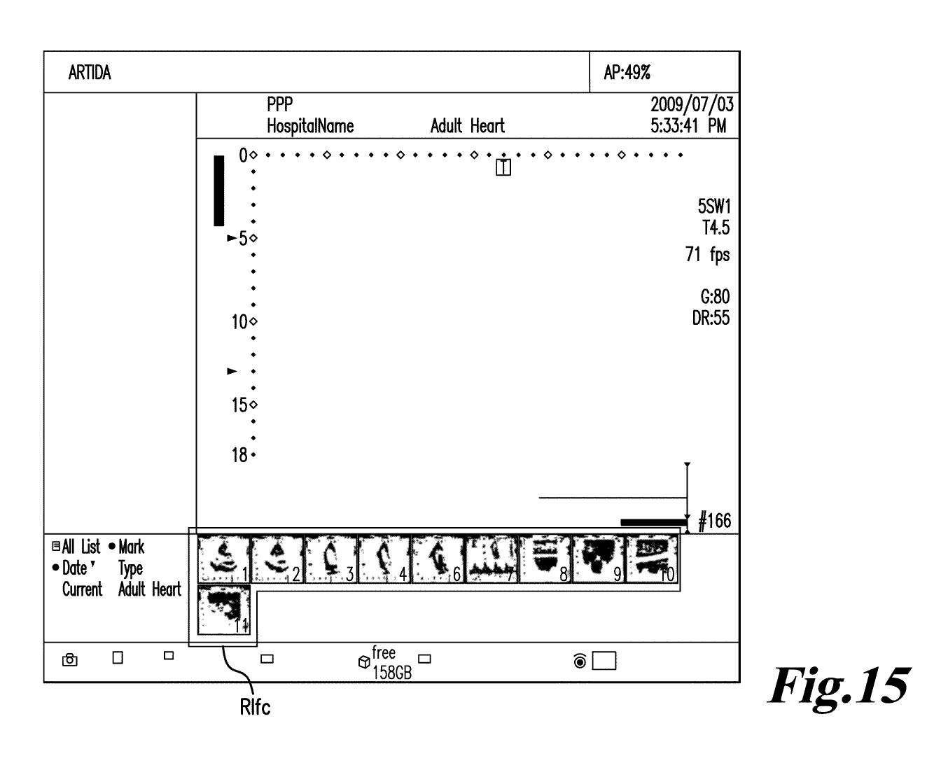

Until the control processor 35 receives an order to stop the ultrasonic scan by the operator, the control processor 35 repeatedly processes the procedure set forth in Step 11 to Step 18 and repeatedly generates the thumbnail composite images. The display unit 47 displays the thumbnail composite images in rows. FIG. 14 shows an example of displaying the thumbnail composite images in rows. Further, FIG. 15 shows an example of conventional system that displays the full-sized composite images of reduced size in rows. The distinction between the display result with and without the additional information generating function described in this embodiment is clarified. In FIG. 15, the operator can barely read off the additional information in the thumbnail image because the additional information in the thumbnail image is displayed too small. But in FIG. 14, the operator can easily read off the additional information in the thumbnail composite image in this embodiment, because the second additional information is displayed relatively bigger than the additional information in FIG. 15.

### First Modified Example

Next, the ultrasonic diagnosis apparatus according to the first modified example will be described using FIG. 2. The difference from the first embodiment is the second additional information. In the first modified example, the second additional information generating unit 43 generates text as the second additional information. The text indicates a time phase according to the ECG waveform. Note that in the first modified example, the second additional information occupies one-sixteenth of the area of the thumbnail composite image.

In FIG. 2, when the control processor 35 receives the order of storing the full-sized composite image to the storage unit 33 from the input unit 45 (Step 13), the first additional information generating unit generates the text. The text indicates the time phase according to the ECG waveform, and indicates when the full-size image was obtained. Next, the second additional information generating unit 43 generates the second additional information by reducing the size of the first additional information (Step 14). For example, the first additional information generating unit 39 generates the text “ED” or “End Diastole”, when the full-size image was obtained within a range of −100 ms to +100 ms of when the E wave was received. As another example, the first additional information generating unit 39 generates the text “ES” or “End Systole”, when the full-size image was obtained within a range of +200 ms to +400 ms of when the R wave was received.

For example, the first additional information generating unit 39 generates the text by reading out information from the storage unit 33. As another example, the text is “ED” and “ES”. The storage unit 33 may store text that is configured by the input operation of the input unit 45.

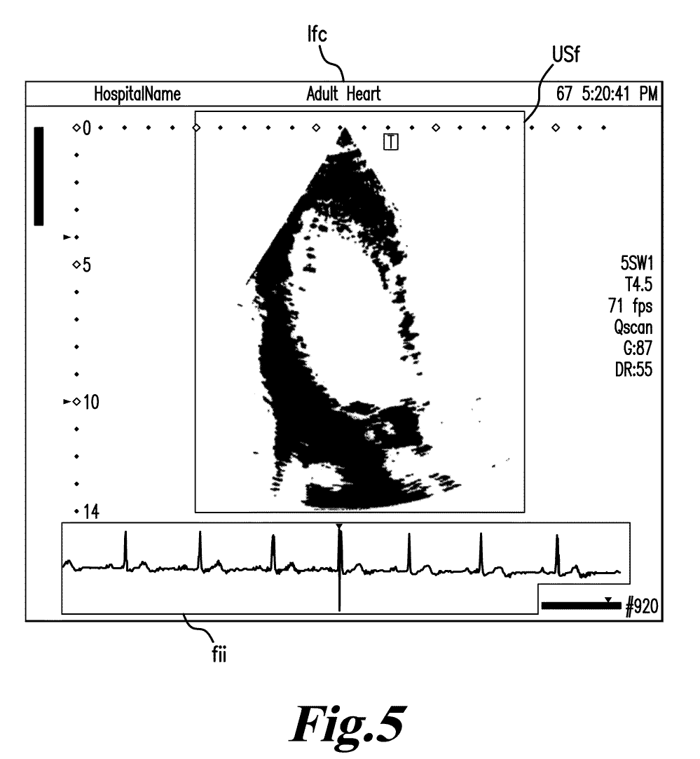

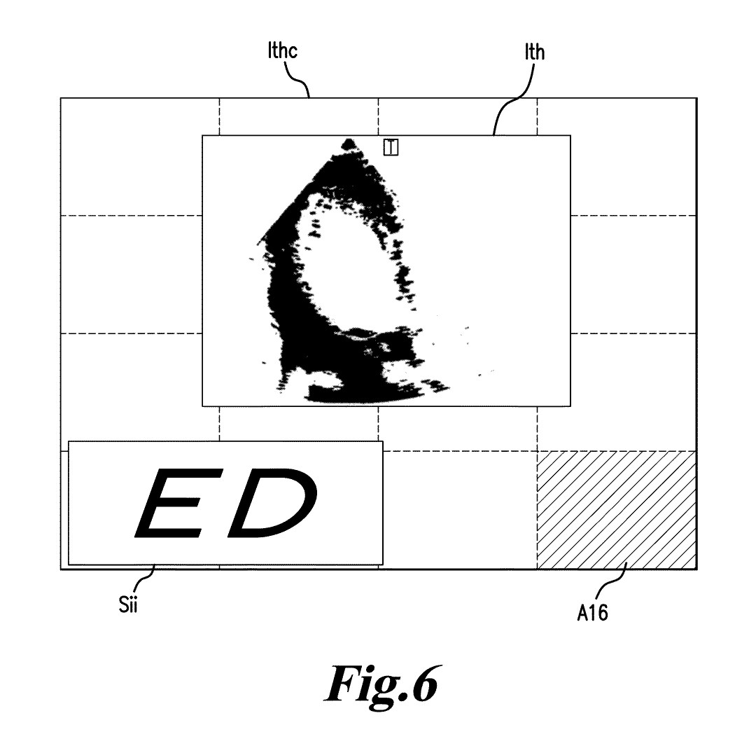

After Step 14, the control unit 35 executes the same procedure described in the first embodiment. FIG. 5 shows an example of the displayed full-sized composite image on the display unit 47. FIG. 6 shows an example of the displayed thumbnail composite image on the display unit 47. In FIG. 6, A16 indicates a one-sixteenth area of the thumbnail image, and Sii indicates the second additional information.

### Second Modified Example

Next, the ultrasonic diagnosis apparatus according to the second modified example will be described using FIG. 2. The difference from the first embodiment is the second additional information. In the second modified example, the second additional information generating unit 43 generates text indicating clinical findings as the second additional information. Note that in the second modified example, the second additional information occupies one-sixteenth of the area of the thumbnail composite image.

In FIG. 2, when the control processor 35 receives the order for storing the full-sized composite image to the storage unit 33 from the input unit 45 (Step 13), the first additional information generating unit generates text indicating the clinical findings. Next, the second additional information generating unit 43 generates the second additional information by reducing a size of the first additional information (Step 14). The first additional information generating unit 39 generates the text by outputting the text from the storage unit 33.

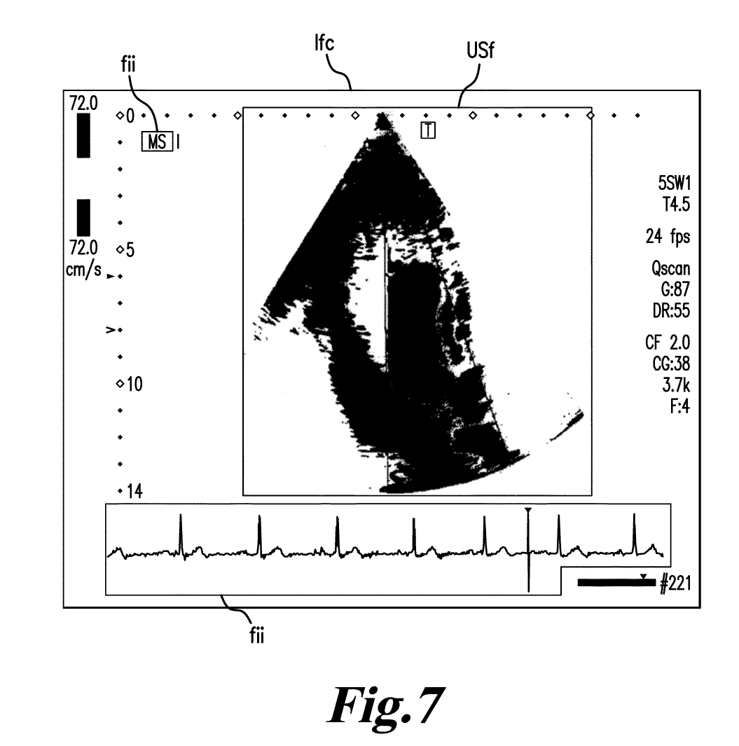

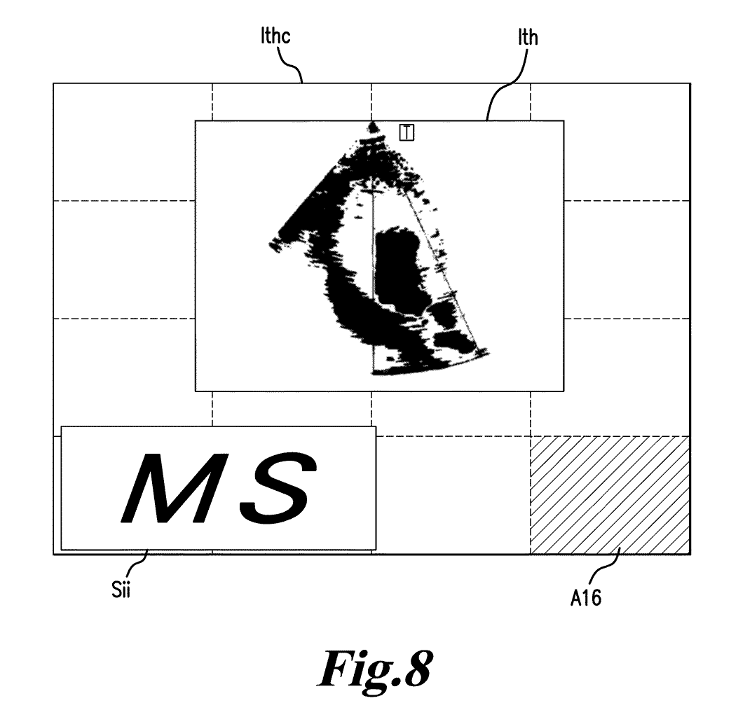

After Step 14, the control unit 35 executes the same procedure described in the first embodiment. FIG. 7 shows the example of the displayed full-sized composite image on the display unit 47. In FIG. 7, fii indicates the first additional information. FIG. 8 shows the example of the displayed thumbnail composite image on the display unit 47, and Sii indicates the second additional information.

### Third Modified Example

Next, the ultrasonic diagnosis apparatus according to the third modified example will be described using FIG. 2. The difference from the first embodiment is the second additional information. In the third modified example, the second additional information generating unit 43 generates a measured value about the Doppler data as the second additional information. Note that in the third modified example, the second additional information occupies one-sixteenth of the area of the thumbnail composite image.

In FIG. 2, when the control processor 35 receives the order for storing the full-sized composite image to the storage unit 33 from the input unit 45 (Step 13), the blood flow measuring unit 37 measures the Doppler data and gets the blood flow information as a value. The blood flow measuring unit 37 outputs the value to the first additional information generating unit 39. For example, the value is blood flow speed. Next, the second additional information generating unit 43 generates the second additional information by reducing a size of the first additional information (Step 14).

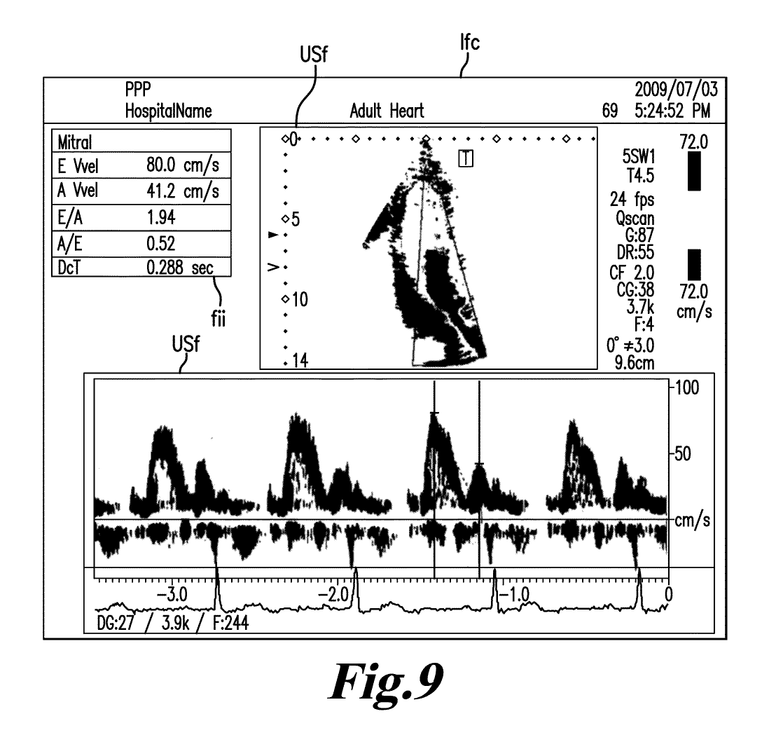

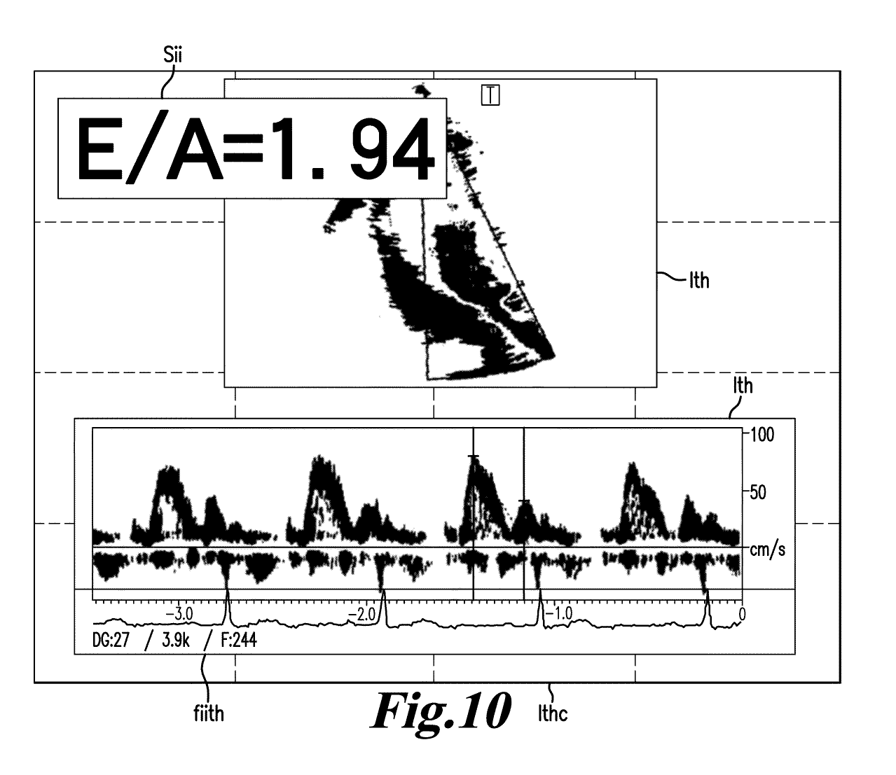

After Step 14, the control unit 35 executes the same procedure described in the first embodiment. FIG. 9 shows the example of the displayed full-sized composite image on the display unit 47. In FIG. 9, USf indicates two full-sized composite images. One of the full-sized composite images is the B-mode image, and another is the Doppler image. In FIG. 9, fii indicates the first additional information. FIG. 10 shows an example of the displayed thumbnail composite image on the display unit 47. In FIG. 10, Sii indicates the second additional information, and faith indicates the ECG waveform that is reduced size using the first size-reduction rate.

### Fourth Modified Example

Next, the ultrasonic diagnosis apparatus according to the fourth modified example will be described using FIG. 2. The difference from the first embodiment is the second additional information. In the fourth modified example, the second additional information generating unit 43 generates a pictogram (body mark) as the second additional information. The pictogram indicates a scan position on the patient's body. Note that in the fourth modified example, the second additional information occupies one-fourth of the area of the thumbnail composite image.

In FIG. 2, when the control processor 35 receives the order for storing the full-sized composite image to the storage unit 33 from the input unit 45 (Step 13), the first additional information generating unit 39 generates the pictogram as the first additional information. Next, the second additional information generating unit 43 generates the second additional information by reducing the size of the first additional information (Step 14).

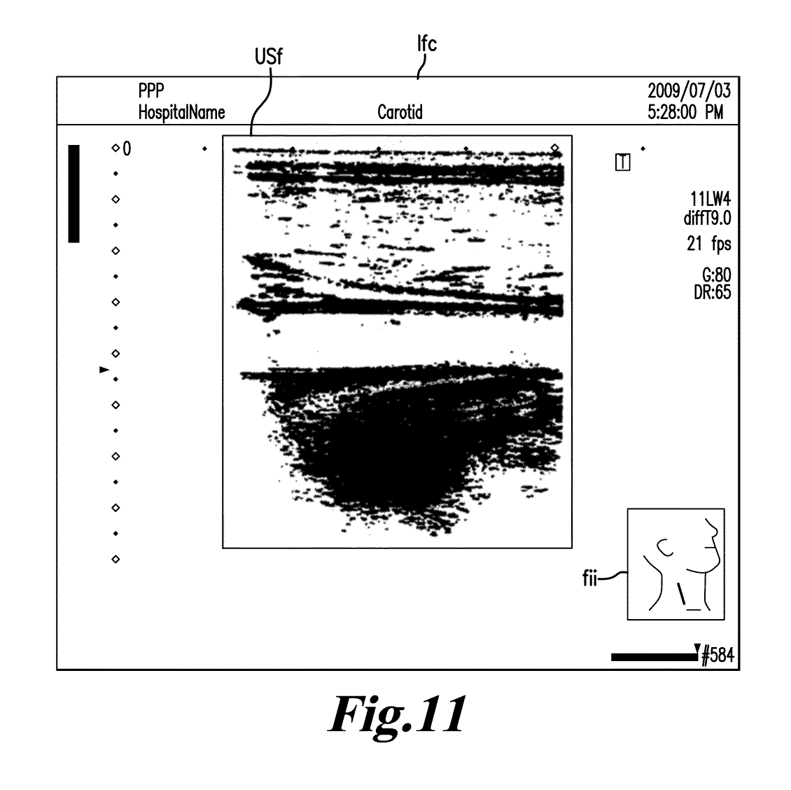

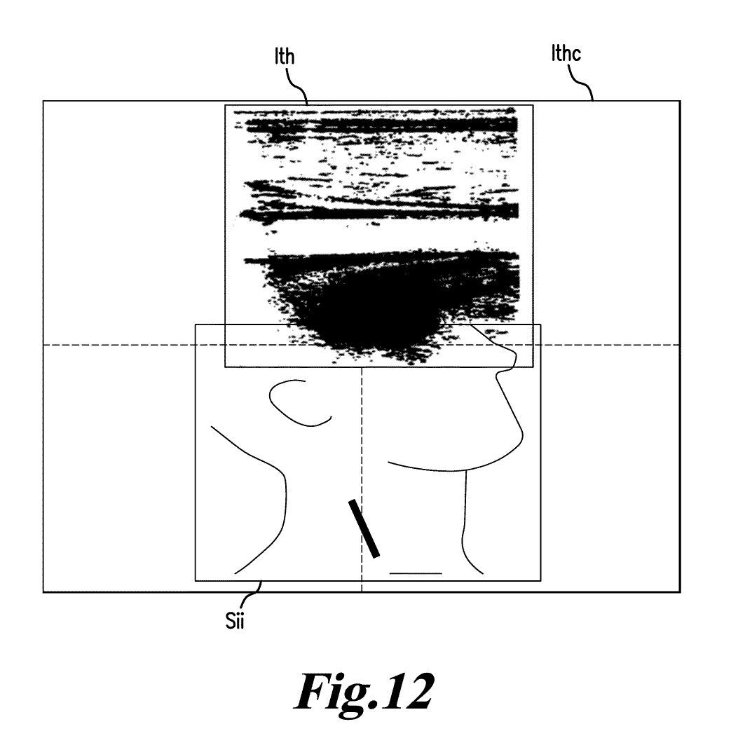

After Step 14, the control unit 35 executes the same procedure described in the first embodiment. FIG. 11 shows the example of the displayed full-sized composite image. In FIG. 11, flu indicates the first additional information. FIG. 12 shows the example of the displayed thumbnail composite image. In FIG. 12, Sii indicates the second additional information. The second additional information occupies one-fourth of the area of the thumbnail composite image. Note that the second size-reduction rate can be changed by an input operation of the input unit 45. Further, a display position of the second additional information in the thumbnail composite image and the type of pictogram can be changed by an input operation of the input unit 45.

### Fifth Modified Example

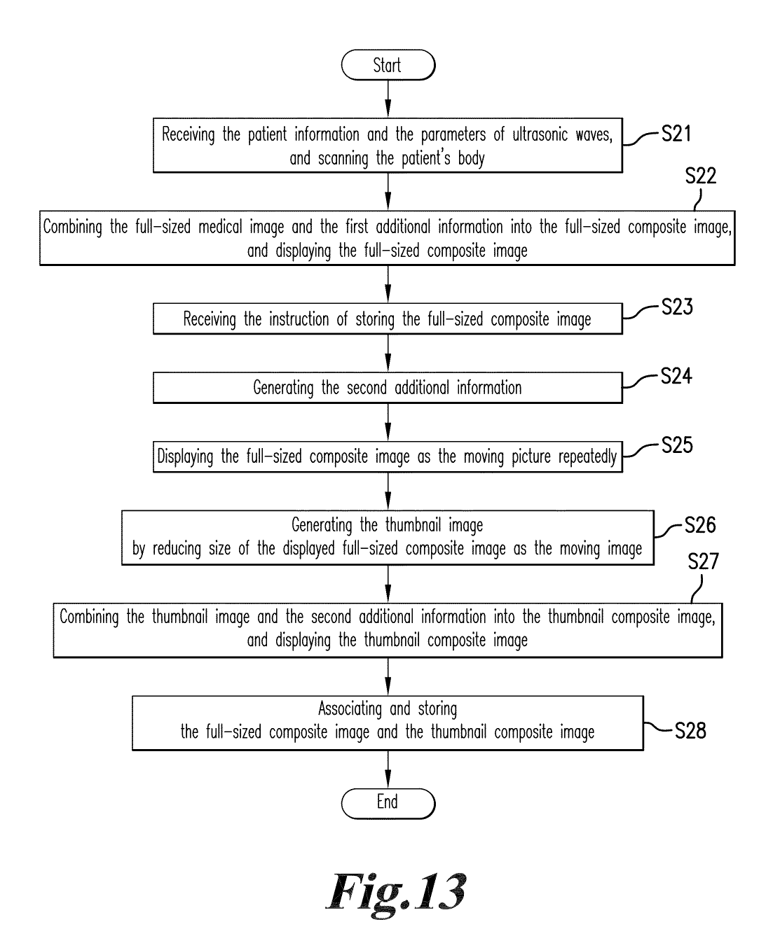

Next, the ultrasonic diagnosis apparatus according to the fifth modified example will be described using FIG. 13. The difference from the first embodiment is the thumbnail image. In the fifth modified example, the ultrasonic image generating unit 27 generates a full-sized medical image as a moving image. Further the thumbnail image generating unit 41 generates the thumbnail image as a moving image.

In FIG. 13, the procedure of Step 21 and Step 22 is the same as Step 11 and Step 12 in FIG. 2. When the control processor 35 receives the order for storing the full-sized composite image as a moving image to the storage unit 33 from the input unit 45 (Step 23), the ultrasonic image generating unit 27 outputs the moving image of a designated period to the image composite unit 29 and the thumbnail image generating unit 41. Further, the first additional information generating unit 39 generates the ECG waveform according to the moving image as the first additional information. The second additional information generating unit 43 generates the second additional information by reducing a size of the first additional information (Step 24).

When the storage unit 33 stores the full-sized composite image, the display unit 47 displays the full-sized composite image as the moving image repeatedly (Step 25). Next, when the storage unit 33 stores the full-sized composite image, the thumbnail image generating unit 41 generates the thumbnail image as a moving image by reducing the size of the full-sized composite image using the second size-reduction rate (Step 26).

The procedure of Step 27 and Step 28 is the same as Step 17 and Step 18 in FIG. 2. The control processor 35 repeatedly executes the procedure described in Step 21 and in Step 28, and repeatedly generates thumbnail composite images. The generated thumbnail composite image is displayed on the display unit 47 as a moving image.

Note that the thumbnail image generating unit 41 may generate the thumbnail image as a still image by extracting the still image from the moving image at a certain time phase. For example, the time phase is the start time of storing the full-sized medical image as the moving image. As another example, the time phase is the time phase configured by an input operation of the input unit 45.

The embodiments described have the following advantages. The image composite unit 29 generates the thumbnail composite image by combining the thumbnail image and the second additional information. The second additional information simplistically represents the first additional information. For example, the second additional information represents the ECG waveform, the PCG waveform, the sphygmograpic waveform, the respiration waveform, the text showing the time phase, the text showing the clinical findings, the text showing value of the Doppler data, or the pictogram. The thumbnail image generating unit 41 generates the thumbnail image by reducing the size of the full-sized medical image using the first size-reduction rate. On the other hand, the second additional information generating unit 43 generates the second additional information by reducing the size of the first additional information using the second size-reduction rate. The first size-reduction rate has a lower value than the second size-reduction rate. That is, the operator can easily read off the clinical information regarding the thumbnail composite image by each reading off the second additional information in the thumbnail composite image.

Accordingly, when the operator tries to display the full-sized composite image at the display unit 47, the operator can read off the second additional information from the thumbnail composite image. Further the operator selects the thumbnail composite image based on the read second additional information. That is, the operator does not need to display full-sized composite images one after another to find the desired full-sized composite image. The operator can refer to the clinical information regarding the thumbnail composite image quickly and easily. This advantage is apparent by comparing the thumbnail composite image (CIthc) in FIG. 14 and the thumbnail image (RIfc) in FIG. 15.

Adaptation to Various Medical Image Processing Apparatuses

The additional information generating function described above can be adapted to various medical image processing apparatuses. For example, the additional information generating function can be adapted to X-ray, CT (Computed Tomography), MRI (Magnetic Resonance Imaging), PET (Positron Emission Tomography), and endscopic instruments, by replacing the components indicated by a dot-dash-line in FIG. 1 with medical image-generating components for the particular medical image processing apparatus. For example, when the additional information generating function is adapted to X-ray, the components indicated by a dot-dash-line in FIG. 1 are replaced by an X-ray generating unit and an X-ray detecting unit.

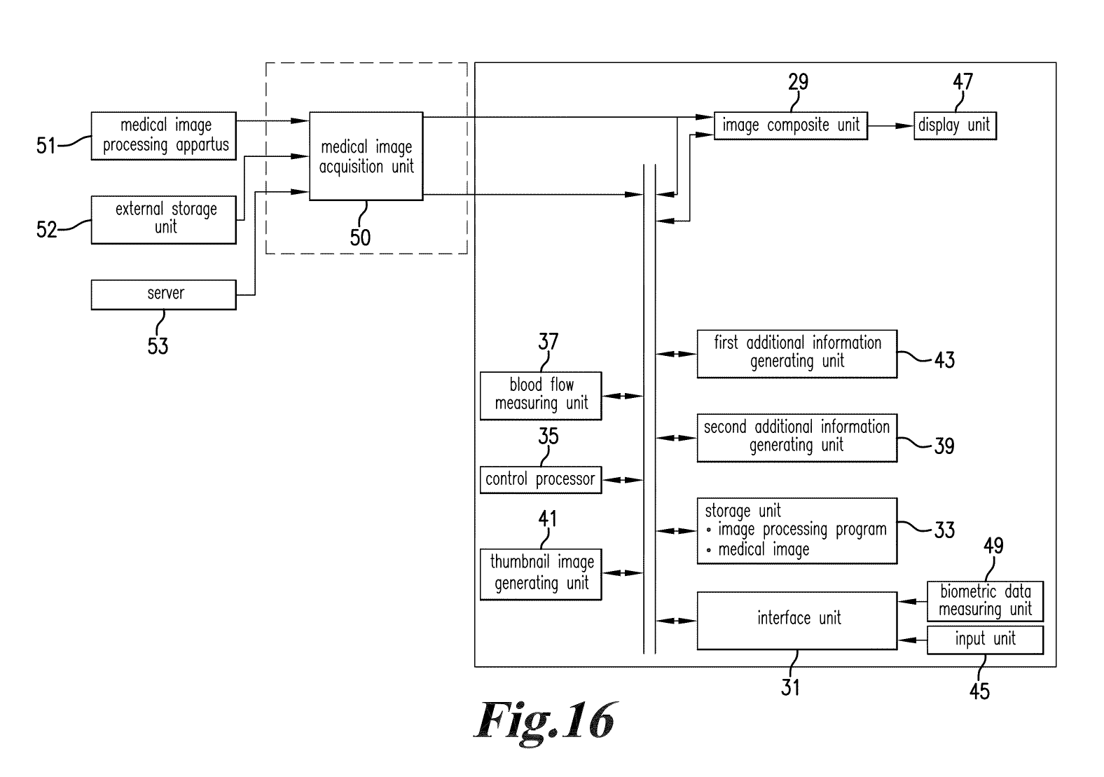

Further, the additional information generating function can be adapted to a workstation for treating and processing medical images using PACS. FIG. 16 shows an example of a workstation adapted to the additional information generating function. In FIG. 16, the workstation is connected to a medical image processing apparatus 51, an external storage unit 52, and a server 53 over a network. A difference between FIG. 16 and FIG. 1 is the medical image acquisition unit 50. As shown in FIG. 16, the workstation includes the medical image acquisition unit 50 instead of the medical image generating components. The medical image acquisition unit 50 is connected the medical image processing apparatus 51, the external storage unit 52, and the server 53 over the network and obtains the medical images as the full-sized images and the first additional information over the network.

The medical image acquisition unit 50 outputs the obtained medical image to the thumbnail image generating unit 41, and the obtained first additional information to the second additional information generating unit 43. The thumbnail image generating unit 41 reduces a display size of the medical image obtained from the medical image acquisition unit 50 using the first size-reduction rate. Further, the second additional information generating unit 43 generates the second additional information by reducing the display size of a part of the first additional information at second size-reduction rate. The image composite unit 29 executes the same procedure described in the first embodiment. Note that the first additional information may be obtained from the first additional information generating unit 39 instead of from the medical image acquisition unit 50.

The respective functions described in the above embodiments and the modified examples can be realized by installing a computer program for executing the processes in a computer such as a workstation and storing them in a memory. The above program, which causes the computer to execute the above processes, can be stored in a recording media such as magnetic disks, hard disks, optical disks, and semiconductor memories, or can be distributed in various memories over a network.

While certain embodiments have been described, these embodiments have been presented by way of example only, and are not intended to limit the scope of the inventions. Indeed, the novel components and systems described herein may be embodied in a variety of other forms. Furthermore, various omissions, substitutions and changes in the form of the components and systems described herein may be made without departing from the spirit of the inventions. The accompanying claims and their equivalents are intended to cover such forms or modifications as would fall within the scope and spirit of the inventions.

1. A medical image processing apparatus, comprising:
a medical image acquisition unit configured to acquire a medical image;
an additional information acquisition unit configured to acquire additional information associated with the medical image;
a thumbnail image composite unit configured to reduce a display size of the medical image using a first size-reduction rate, to reduce a display size of the additional information using a second size-reduction rate, and to combine the size-reduced medical image and the size-reduced additional information into a thumbnail composite image; and
a display unit configured to display the thumbnail composite image.

2. The apparatus according to claim 1, further comprising:
an input unit configured to receive an input operation; and
a storage unit configured to store the medical image,
wherein the thumbnail image composite unit is configured to combine the size-reduced medical image and the size-reduced additional information into the thumbnail composite image when the thumbnail image composite unit receives a first input operation that causes the medical image to be stored in the storage unit or a second input operation that causes the medical image to be read out from the storage unit.

3. The apparatus according to claim 1, further comprising:
an input unit configured to receive an input operation,
wherein said additional information is text based on said input operation, which is acquired by said additional information acquisition unit.

4. The apparatus according to claim 1, wherein the additional information acquisition unit is configured to acquire the additional information based on biological signals, and the additional information is text representing a time phase, an electrocardiography waveform, a phonocardiography waveform, a sphygmographic waveform, or a measured value of the biological signals.

5. The apparatus according to claim 1, wherein the additional information acquisition unit is configured to acquire the additional information, which is a pictogram representing a position at which the medical image was acquired.

6. The apparatus according to claim 1, wherein the medical image acquisition unit is configured to acquire the medical image, which is an X-ray image, an X-ray computed tomography image, a magnetic resonance image, an ultrasound image, a positron emission tomography image, an endscopic image, a 3D medical image, a moving medical image, or a composite medical image.

7. A medical image processing method, comprising:
obtaining a medical image;
obtaining additional information associated with the medical image;
reducing a display size of the medical image using a first size-reduction rate;
reducing a display size of the additional information using a second size-reduction rate;
combining the size-reduced medical image and the size-reduced additional information into a thumbnail composite image;
displaying the thumbnail composite image.

8. The method according to claim 7, further comprising:
receiving an input operation; and
storing the medical image,
wherein the thumbnail composite image is combined when an input operation that causes the medical image to be stored is received.

9. The method according to claim 7, further comprising:
receiving an input operation; and
reading out the stored medical image,
wherein the thumbnail composite image is combined when an input operation that causes the stored medical image to be read out is received.

10. The method according to claim 7, further comprising:
receiving an input operation, wherein the additional information is text based on the input operation.

11. The method according to claim 7, wherein the additional information is acquired based on biological signals, and the additional information is text representing a time phase, an electrocardiographic waveform, a phonocardiographic waveform, a sphygmographic waveform, or a measured value of the biological signals.

12. The method according to claim 7, wherein the additional information is a pictogram representing a position at which the medical image was acquired.

13. The method according to claim 8, wherein the medical image is an X-ray image, an X-ray computed tomography image, a magnetic resonance image, an ultrasound image, a positron emission tomography image, an endscopic image, a 3D medical image, a moving medical image, or a composite medical image.

14. An ultrasonic diagnosis apparatus that executes ultrasonic scanning using an ultrasonic probe, the ultrasonic diagnosis apparatus comprising:
an ultrasonic image acquisition unit configured to acquire an ultrasonic image by executing ultrasonic scanning;
an additional information acquisition unit configured to acquire additional information associated with the ultrasonic image;
a thumbnail image composite unit configured to reduce a display size of the ultrasonic image using a first size-reduction rate, to reduce a display size of the additional information using a second size-reduction rate, and to combine the size-reduced ultrasonic image and the size-reduced additional information into a thumbnail composite image;
a display unit configured to display the thumbnail composite image.

15. The ultrasonic diagnosis apparatus according to claim 14, further comprising:
an input unit configured to receive an input operation; and
a storage unit configured to store the ultrasonic image,
wherein the thumbnail image composite unit combines the size-reduced ultrasonic image and the size reduced additional information into the thumbnail composite image when the thumbnail image composite unit receives an input operation that causes the ultrasonic image to be stored in an storage unit or an input operation that causes the ultrasonic image to be read out from the storage unit.

16. The apparatus according to claim 14, further comprising:
an input unit configured to receive an input operation,
wherein the additional information is text based on the input operation acquired by the additional information acquisition unit.

17. The apparatus according to claim 14, wherein the additional information acquisition unit acquires the additional information based on biological signals, and the additional information is text representing a time phase, an electrocardiographic waveform, a phonocardiographic waveform, a sphygmographic waveform, or a measured value of the biological signals.

18. The apparatus according to claim 14, wherein the additional information acquisition unit is configured to acquire the additional information, which is a pictogram representing a position of ultrasonic scanning.

19. The apparatus according to claim 14, wherein the ultrasonic image is a B-mode image, a Doppler image, an erastgraphy image, a M-mode image, a 3D ultrasonic image, a moving ultrasonic image, or a composite ultrasonic image.
